(19) United States
(12) Patent Application Publication
(10) Pub. No.: US 2026/0345936 A1
(43) Pub. Date: Nov. 5, 2026

(54) Device and Method for Dynamically Sealing Occlusion or Space-Filling Tamponade of a Hollow Organ

(71) Applicant: Advanced Medical Balloons GmbH, WAGHÄUSEL (DE)

(72) Inventor: Fred Göbel, Speyer (DE)

(21) Appl. No.: 19/743,471

(22) Filed: Jul. 15, 2026

Related U.S. Application Data
(63) Continuation of application No. 17/533,691, filed on Nov. 23, 2021, now Pat. No. 12,734,320.
Application No. 17/533,691 is a continuation-in-part of application No. 15/533,151, filed on Dec. 4, 2015, now Pat. No. 11,207,482.

(30) Foreign Application Priority Data
Dec. 4, 2014 (DE) 10 2014 017 872.2
Jan. 22, 2015 (DE) 10 2015 000 621.5
Jan. 29, 2015 (DE) 10 2015 001 030.1
Mar. 4, 2015 (DE) 10 2015 002 995.9
Nov. 18, 2015 (DE) 10 2015 014 824.9

(51) Int. Cl. A61M 16/04; A61M 25/10

(52) U.S. Cl. CPC A61M 16/044; A61M 16/0486; A61M 25/10186

(57) ABSTRACT

A device for dynamically sealing intubation of a hollow organ is disclosed. The device includes a shaft-shaped tube configured to be inserted into the hollow organ. The shaft-shaped tube has a primary lumen that provides access through the hollow organ or to the hollow organ. The device includes an intracorporeal sealing balloon that surrounds a distal region of the shaft-shaped tube. The intracorporeal sealing balloon is configured to operate as a cuss that dynamically seals the hollow organ. One or more secondary lumens for filling the intracorporeal sealing balloon are integrated into a wall of a proximal region of the shaft-shaped tube.

### CROSS-REFERENCE TO RELATED APPLICATIONS

The present application claims priority to U.S. patent application Ser. No. 17/533,691, filed Nov. 23, 2021, which is hereby incorporated by reference in its entirety. U.S. patent application Ser. No. 17/533,691 claims priority, and is a continuation-in-part, to U.S. Pat. No. 11,207,482, issued Dec. 28, 2021, which is hereby incorporated by reference in its entirety. U.S. Pat. No. 11,207,482 is a national stage entry of International Application No. PCT/IB2015/002309, filed Dec. 4, 2015, which claims the benefit of priority to German Patent Application No. DE 10 2014 017 872.2, filed Dec. 4, 2014, German Patent Application No. DE 10 2015 000 621.5, filed Jan. 22, 2015, German Patent Application No. DE 10 2015 001 030.1, filed Jan. 29, 2015, German Patent Application No. DE 10 2015 002 995.9, filed Mar. 4, 2015, and German Patent Application No. DE 10 2015 014 824.9, filed Nov. 18, 2015, the entire disclosure of which is incorporated herein by reference.

### FIELD OF THE DISCLOSURE

The invention is directed to a device and a method for the dynamic occlusion or tamponade of a hollow organ by means of a balloon-like element, in particular for the dynamic, aspiration-preventing seal of the intubated trachea in patients who are breathing independently and in patients in a machine-assisted spontaneous ventilation mode.

### BACKGROUND

In many cases, the continuous motility of the organ itself is a fundamental problem associated with the sealing closure or space-filling tamponade of organs or cavities with a fillable, balloon-like element in a way that is gentle to the organ and efficient. Organs or body cavities that are limited by musculo-connective tissue often have autonomous motility or are subjected to the dynamics of adjacent organs or structures. In order to create a continuous seal of the organ lumen, autonomous or correspondingly motile organs require a particular regulating mechanism that reacts quickly to fluctuations in the organ diameter or changes in the tone of the organ wall. As the changes in diameter or tone occur, said mechanism must act as a synchronously as possible.

The problem of dynamic, synchronized adaptation of a balloon occlusion of a hollow organ can be illustrated with the example of the human trachea. The trachea is a tube-like structure with portions composed of cartilaginous tissue, connective tissue and muscle. It extends from the lower section of the larynx to the branching of the main bronchi. The front and side portions of the trachea are stabilized by clip-like, approximately horseshoe-shaped structures, which in turn are connected to each other in the longitudinal direction by connective tissue-like layers of tissue. The trachea lumen is closed off on the rear-wall side by the so-called pars membranacea, which consists of material made of continuous musculo-connective tissue, without any reinforcing cartilaginous elements. The esophagus, which is made of musculo-connective tissue, lies on its dorsal side.

The upper quarter of the trachea is usually located outside of the thorax, while the two lower three quarters lie within the thoracic cavity delimited by the thorax and diaphragm. The lower thoracic portion of the trachea is thereby especially subjected to the pressure fluctuations in the thoracic cavity that occur as part of the thoracic respiratory mechanics of a spontaneously breathing patient as well as a patient who is spontaneously breathing with assistance.

When the patient breathes in, the thoracic volume is enlarged by the lifting of the ribs and simultaneous lowering of the diaphragm, and the intrathoracic pressure therefore decreases” As a result of respiratory mechanics, this drop in pressure leads to the inflow of inhaled air into the lungs, which expands with the increase in thoracic volume.

However, the pressure drop in the thorax associated with the increase in thoracic volume also leads to corresponding drops in pressure within filled balloon elements positioned in the patient's thorax. Balloon elements such as these are used in tracheal tubes and tracheostomy cannulas, for example, to seal the deep airways against inflowing secretions from the throat and to permit positive pressure ventilation of the patient's lungs. The cyclical drops in thoracic pressure caused by the patient's own breathing can move the sealing pressure in the balloons of ventilation catheters into low ranges, in which a sufficient seal against secretions that collect in the trachea above the sealing balloon (cuff) is no longer ensured. See Badenhorst C H, Changes in tracheal cuff pressure in respiratory support, Grit Care Med, 1987; 15/4:300-302.

Whereas the sealing performance of conventional PVC tracheal tube cuffs correlates very closely with the effective filling pressure in the cuff, the especially thin-walled polyurethane tracheal tube cuffs demonstrate significantly more stable sealing efficiency when the tracheal sealing pressures fall into ranges around 15 mbar. See Bassi G L, Grit Care Med, 2013; 41:518-526.

Of particular importance for the secretion seal of tracheal sealing balloons, however, is the pressure range from 5 to 15 mbar, which is easily achieved during the independent breathing of a patient, essentially from breath to breath. Seal-optimized ventilation catheters with micro-thin-walled PUR cuffs can also still ensure good sealing performance at ca. 10 mbar fining pressure. However, they cannot protect against inflowing, infectious secretions during pressure drops below 10 mbar to sub-atmospheric thoracic pressure values.

No sealing technology is yet available for sealing the trachea by means of a cuff-like sealing balloon, which synchronizes with the breathing effort of the thorax, is atraumatic, seals efficiently over sufficiently wide ranges of fining pressure and is cost-effective to manufacture. Although many different technical embodiments of filling pressure-regulating devices for tracheal ventilation catheters are described in the prior art, a sufficiently synchronized adaptation of the sealing pressure to alternating thoracic pressures, as are observed in autonomous breathing in patients, has not yet been achieved.

In the known ventilation catheters, the trachea-sealing balloon element is usually filled by small-bore filling lines which are extruded into the shaft of the catheter. The small cross-sections of the filling lines generally do not ensure a flow rate of the filling medium which fills the balloon that is sufficiently strong to maintain a tracheal seal during the drop in thoracic pressure. Even technically complicated extracorporeal control mechanisms, such as the CDR 2000 device produced by Logomed GmbH (no longer commercially available), function inadequately because of the small-bore supply lines between the sealing balloon element and the regulator.

### SUMMARY

The present invention describes a novel catheter technology that permits a quick flow rate between a regulating mechanism outside the body and a balloon-like sealing element placed in the trachea by means of an especially flow-efficient supply line for the filling medium. Technologically simple and cost-effective means are used to compensate for the cuff volume in a way that is sufficiently synchronized for the dynamic sealing of the trachea of a spontaneously breathing patient and that maintains the seal.

Especially, the invention describes a device for the dynamically sealing intubation of a hollow organ, comprising a tube in the form of a shaft that can be inserted into the hollow organ, with a primary lumen to provide access through or to the hollow organ in question, and comprising an intracorporeal sealing balloon, which surrounds a distal region of the shaft of said tube in the manner of a cuff for the purpose of sealing it against the hollow organ, wherein one or more secondary lumens for filling said intracorporeal sealing balloon are integrated into the wall of at least a proximal region of said shaft, wherein, within each cross-sectional plane that is intersected perpendicularly by the local longitudinal direction of the device, the following applies for the overall interior cross-section Q1 of the primary lumen and the sum Q2 of the interior cross-sections of all secondary lumens:

Q2/(Q1+Q2)≥0.06,

wherein at an extracorporeal filling tube, which communicates with all secondary lumens, a control device is provided in order to keep the pressure within the intracorporeal sealing balloon nearly constant in such a way that
a) when the volume of the hollow organ increases, a corresponding amount of the filling medium is forced to flow into the intracorporeal sealing tube in order to increase the volume of the intracorporeal sealing tube accordingly, and
b) when the volume of the hollow organ decreases, a corresponding amount of the filling medium is allowed to flow out of the intracorporeal sealing tube in order to decrease the volume of the intracorporeal sealing tube accordingly.

The invention further describes a thin-walled ventilation catheter shaft that has a single lumen and a shaft wall that is preferably stabilized by a corrugated tube-like corrugation. The corrugation of the shaft permits especially low wall thicknesses, which optimally allows for large inner lumens for the particularly low-resistance breathing of the patient. The corrugation additionally gives the shaft a particular low-tension or non-elastic axial flexibility and thus adaptability in the trachea of a spontaneously breathing patient. A flexible shaft such as this can additionally be made of especially hard PUR types, which then permit particularly thin wall thicknesses.

One possible embodiment of the invention is based on a balloon element which is fixedly and sealingly attached to the supporting shaft body at the distal end of the ventilation catheter and the proximal end of which narrows almost to the outer dimensions of the catheter shaft, but leaves a coaxial gap for the shaft. This proximal extension of the balloon can extend into the thoracic section of the trachea, but can also be guided into the extra-thoracic sections of the trachea as far as the region of the vocal cords or also to the lower region of the throat, wherein a particular free gap is formed between the sleeve of the proximal balloon segment that narrows in this way and the enclosed shaft.

The gap created in this way permits an especially large-bore, efficient volume flow between a volume reservoir disposed outside the body and the trachea-sealing balloon element. The gap further prevents potentially damaging direct contact by the corrugated catheter shaft with the epithelium of the trachea.

The concentric arrangement of the catheter shaft in a tapering, tubelike proximal balloon-segment is advantageous especially in the region of the vocal cords. Here, the balloon envelope that encloses the shaft rests against the vocal cords in a protective way and prevents abrasive effects by the shaft when it moves relative to the respiratory tract.

In the proximal section of the tracheal ventilation catheter, the end of the tube-like tapered balloon segment is preferably accommodated by a multi-lumen shaft element. This shaft element preferably has a supply line cross-section whose flow-affecting cross-section preferably corresponds to the flow-affecting cross-section of the gap between the shaft and the proximal balloon segment. A filling tube then attaches to the supply lumen integrated into the proximal catheter, said filling tube having a diameter with corresponding flow characteristics to those ensured within the catheter.

The invention shows an example of a method of calculating the dimensions of the claimed gap created between the catheter shaft and the proximal extension of the balloon end or balloon body, which provides a good approximation of how large the radial gap must be in order to achieve a sufficiently compensating volume flow between the regulator and balloon in under 10 milliseconds after thoracic pressure begins to decrease.

Both the proximal balloon segment surrounding the shaft and the distal balloon segment sealing the trachea are preferably produced from a material that is as thin-walled as possible and only slightly elastic. In this way, the required dimensional stability of the balloon body is ensured when it is loaded with filling pressure from the inside or under mechanical stress from the outside.

The invention further describes various methods for quickly sealing the trachea in a volume-compensating way, wherein a claimed catheter provided with an occluding or tamponading balloon element is connected to an extracorporeal regulator device. By coupling the catheter with the regulator, an interior space is created that can be charged with isobaric filling pressure.

The continuously flow-efficient, large-bore connection between the sealing balloon element and a volume source in which the pressure is kept constant represents the required synchronicity between the patient's thoracic respiratory activity and the volume flow that is needed to obtain the tracheal seal.

Quick shifts in volume are permitted in the claimed combination of a catheter and a regulator, e.g. by gravity- or spring-powered regulators, which function in the range of low, physiologically harmless pressures, i.e. in a pressure range of ca. 25 to 35 mbar, and do not require unphysiologically high pressure gradients that force the volume flow in order to ensure sufficient volume flows over the connecting supply line between the cuff and regulator.

The regulator can be configured according to the design described in PCT/EP/2013/056169, for instance. The present invention is preferably designed for this simple form of control by a communicating system of interior spaces under isobaric pressure in the physiological pressure range of 20 to 50, preferably 20 to 35 mbar.

The claimed optimized volume flow between the trachea-sealing balloon body and the extracorporeal regulator can also be achieved with tracheal tubes and tracheostomy cannulas of a conventional design, insofar as the supplying feed channel has a flow-affecting cross-sectional area that corresponds to a circular cross section with a circular diameter of at least 2 mm and preferably a circular diameter of 4 to 6 mm. In products with a convention design, the volume-supplying channel is usually circular and has an inner diameter of ca. 0.5 to 0.7 mm. The channel is extruded into the shaft wall of the tube shaft. It transitions outside the shaft into a tube line, which is normally connected to a one-way valve. For the purpose of the thinnest-walled shafts possible and/or in order to permit the largest possible inner diameter of the ventilation lumen, the supply lines in the shaft wall are kept correspondingly low-caliber.

The principle of flow-optimized volume compensation described above can similarly be applied to the dynamic tamponade of the esophagus of a spontaneously breathing patient. The pressure fluctuations in a tamponade balloon placed in the esophagus, which correspond to independent breathing, are usually more pronounced than in a balloon placed in the trachea. They generally conform to the prevailing absolute intrathoracic pressure. To produce a balloon tamponade in the esophagus that seals as efficiently as possible, it is proposed that the balloon be segmented so that it corresponds to the trachea. While the distal segment of the balloon, which actually seals the esophagus, extends over the extent of the esophagus between its upper and lower sphincters, a tapering balloon segment attaches in the proximal direction and optionally extends to the proximal end of the catheter supporting the balloon. The diameter ratios that are hereafter represented on the ventilation catheter and that define the resulting gap between the shaft and the proximal balloon segment can also be applied in the case of gastric tubes for nutrition and/or decompression. Here, too, the shaft of the gastric tube can consist of a single-lumen, thin-walled tube that is corrugated, entirely or in segments, to improve the bending mechanics. The connection to an isobaric volume reservoir can occur in the manner according to the invention, similarly to the tracheal tube.

### BRIEF DESCRIPTION OF THE DRAWINGS

Further features, details, advantages and effects based on the invention arise from the following description of preferred embodiments of the invention and with the aid of the accompanying drawings.

FIG. 1 shows a claimed tracheal tube in combination with an external volume-regulating device.

FIG. 2 shows the volume-shafting gap S relative to the overall diameter G in the vicinity of the proximal balloon extension.

FIG. 3 shows a tracheal tube with multiple volume-shifting supply lines arranged in or on the catheter shaft.

FIG. 3a shows a cross-section of the supplying, shaft-integrated lumens of the tube described in FIG. 3.

FIG. 4 shows a particular embodiment of the tube shown in FIG. 1, in which the trachea-sealing balloon extends beyond the glottis plane.

FIG. 5 shows an illustrative embodiment of a claimed tracheostomy cannula.

FIG. 6 shows a claimed tracheal tube with a sensor element in the region of the trachea-sealing balloon segment and a regulator unit that is arranged with a sensor in a control loop.

FIG. 7 shows a combined valve/throttle mechanism which prevents the balloon from quickly emptying into the regulator/reservoir, which would be critical for the seal.

FIG. 8 shows a gastric tube with a proximally extended balloon segment for the dynamically sealing tamponade of the esophagus in combination with an extracorporeal isobaric volume reservoir.

FIG. 9 shows another embodiment of the present invention in a representation according to FIG. 6.

FIG. 10 shows still another embodiment of the present invention in a representation according to FIG. 6.

FIG. 11a shows the characteristic equation of a PD controller which can be used together with the present invention.

FIG. 11b shows the electronic circuit diagram of the PD controller which has the characteristic equation of FIG. 11a.

FIG. 11c shows the step response of the PD controller with the electronic circuit according to FIG. 11b.

FIG. 11d shows the relation between the parameters of the characteristic equation according to FIG. 11 a and the elements of the electronic circuit according to FIG. 11b.

### DETAILED DESCRIPTION

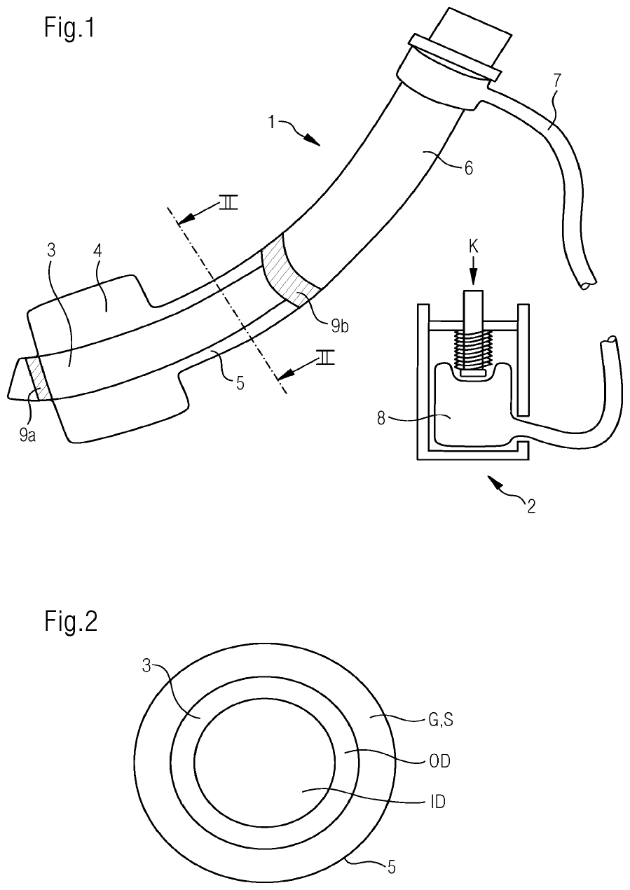

FIG. 1 describes, in an illustrative total overview, the communicating connection/coupling of a claimed device 1, in the form of a tracheal tube, with a preferably gravity- or spring-driven, isobaric volume reservoir 2.

The supply chamber formed by the freely communicating coupling of the tracheal tube and volume reservoir 2 consists of the trachea-sealing balloon segment 4, the tube-like tapered balloon end 5 that connects in the proximal direction, the supply lumen{s) in the proximal shaft element 6, the flexible supply line 7 to the reservoir that attaches to the shaft as well as the reservoir volume 8 of the regulator.

The distal shaft segment of the tube 3 supports a trachea-sealing balloon 4 at its distal end, said balloon being sealingly attached at its distal end 9a to the surface of the shaft. A tube-like proximal balloon segment 5, which tapers relative to the diameter of the sealing balloon segment 4, attaches to the balloon segment 4 in the proximal direction. Its proximal end locks tightly to the surface of the distal end 9b of the proximal shaft element 6.

If a decrease in intrathoracic pressure occurs during the course of inhaling (inspiration), and thus a corresponding transient widening of the tracheal cross-section, which in turn causes a corresponding drop in pressure within the balloon body placed in the trachea, volume flows from the reservoir 2 to the sealing balloon segment 4, wherein the reservoir continuously charges the volume with a defined pressure. As a result, the tracheal sealing pressure can be maintained even when the patient inhales deeply, with a possible pressure drop in the thorax and/or in the trachea-sealing balloon to sub-atmospheric levels, without relevant losses in the sealing capacity.

In a preferred embodiment, the reservoir 2 consists of a reservoir body 8, which can be configured e.g. as a balloon or bellows, and establishes a constant isobaric pressure in the supply chamber by a force K acting on the reservoir.

Crucial to achieving the smallest possible time latency between the initial widening of the tracheal cross-section or the initial reduction of the transmural thoracic forces acting on the tracheal sealing balloon and the start of a seal-creating shift of filling medium to the trachea-sealing balloon segment is, above all, the flow-affecting cross-sectional area of the gap S available the between the distal shaft segment 9 and the proximal extension 5 of the balloon.

In the following figure, the invention proposes especially advantageously dimensioned ratios of the cross-sectional area S to the cross-sectional area of the ventilation lumen ID and to the overall cross-sectional area OD of the catheter between the proximal shaft 6 and the trachea-sealing balloon segment 4.

FIG. 2 shows a cross-section through the volume-supplying segment 5 of the balloon of the tracheal tube pictured in FIG. 1, said segment attaching to the trachea-sealing balloon in the proximal direction.

S designates the gap surface that is preferred for the supply of filling medium to the balloon. It is defined as the difference between the cross-sectional area G, which is delimited by the sleeve wall of the supplying balloon end 5, and the cross-sectional area OD of the shaft body, which is delimited by the outer surface of the shaft. Here the cross-sectional area S should be 1/10 to 5/10 of cross-sectional area G, especially preferably 2/10 to 3/10 of cross-sectional area G.

Relative to the cross-sectional area ID of the inner lumen of the shaft body, cross-sectional area S should amount to 2/10 to 6/10 of cross-sectional area ID, especially preferably 3/10 to 4/10 of cross-sectional area ID.

In addition to air as the preferred medium, liquid media can also be used to fill the trachea-sealing system.

For the quantitative calculation of the flow conditions in the volume-conducting interior space of the balloon, in particular based on the pressure ratios in the trachea-sealing balloon segment 4, the following place-holder values should be used:
Vi Volume of the distal balloon segment 4
Pi Pressure in the distal balloon segment 4
Pi Filling density in the distal balloon segment 4
M₁ Air mass in the distal balloon segment 4
V₂ Volume of the extracorporeal reservoir 8
P₂ Pressure in the extracorporeal reservoir 8
p₂ Fining density in the extracorporeal reservoir 8
m₂ Air mass in the extracorporeal reservoir 8

The following applies for air masses m−1, m2:

$${m}_{1} ( t ) = {m}_{1 , 0} + {\int }_{τ = 0}^{τ = t} {S}_{m , 1} ( τ ) d τ \qquad ( 1 )$$

$${m}_{2} ( t ) = {m}_{2 , 0} - {\int }_{τ = 0}^{τ = t} {S}_{m , 2} ( τ ) d τ \qquad ( 2 )$$

S_{m,v} stands for the air flow to the respective balloon 4, 8 as an air mass flow.

According to the Hagen-Poiseuille equation, the following is true for the mass fluid flow through a line with a circular cross-section and with an inner radius R and length L:

$${S}_{m , 1} = \frac{{ρ}_{1} \cdot π ( {p}_{2} - {p}_{1} ) \cdot {R}^{4}}{8 η \cdot I} \qquad ( 3 a )$$

$${S}_{m , 2} = \frac{{ρ}_{2} \cdot π ( {p}_{2} - {p}_{1} ) \cdot {R}^{4}}{8 η \cdot I} \qquad ( 3 b )$$

If, however—as in this case—the secondary lumen represents an annular structure around a primary lumen, then the Hagen-Poiseuilie formula does not exactly apply.

Instead, one must consider a space with a strip-like cross-section, which can ideally be imagined in a straightened form, i.e. having a flat structure or a rectangular cross-section with length L and thickness D, i.e. with a cross-sectional area Q=L·D.

Between two plates at a distance D, the following applies for the distribution of the flow rate v(x) along a direction x perpendicular to the plates:

$$v ( x ) = \frac{( {p}_{1} - {p}_{2} ) \cdot ( {D}^{2} / 4 - {x}^{2} )}{2 η \cdot I} \qquad ( 4 )$$

This is a parabolic curve. By integration over cross-sectional area Q, the mass flowing through cross-sectional area Q during time t can be determined:

$${S}_{m , 1} = \frac{{ρ}_{1} \cdot ( {p}_{2} - {p}_{1} ) \cdot L \cdot {D}^{3}}{12 η \cdot I} \qquad ( 5 a )$$

$${S}_{m , 2} = \frac{{ρ}_{2} \cdot ( {p}_{1} - {p}_{2} ) \cdot L \cdot {D}^{3}}{12 η \cdot I} \qquad ( 5 b )$$

In any case, these formulas replace the above Hagen-Poiseuille formulas (3a) and (3b) for annular balloon segment 5.

Here η stands for the dynamic viscosity of the flowing gas. For air:
η is 17.1 μPa·s at 273 K.
   Furthermore, because of the thermal equation of state of ideal gases, the following applies in the balloon 4:

η₁=ρ₁·R_{S}·T₁ (6a)

and in balloon 8:

η₂=ρ₂·R_{S}·T₂ (6b)

In this case, Rs is the individual or specific gas constant, which for air has the value 287.058 J/(kg*K). Tv is the temperature in balloon sections 4 and 5 and in the balloon 8.

For a temperature of 23 °C or 296 K, the factor

k=R_{S,air}·T_{23 °C}=85·10³ J/(kg·K)

It should be assumed hereafter that the temperature both in balloon 4 and in balloon 8 is I constant 23 °C.

T₁=T₂=296 K.

Then the following applies:

ρ₁=p₁·k (8)

p₂=ρ₂·k (9)

Thus, by inserting equation (5a) into equation (1), the result is:

$${m}_{1} ( t ) = {m}_{1 , 0} {\int }_{τ = 0}^{τ = t} \frac{{ρ}_{1} \cdot L \cdot {D}^{3}}{{12}_{η \cdot I}^{( 10 )}} ( {p}_{2} - {p}_{1} ) d τ$$

With equation (8), it follows that:

$${m}_{1} ( t ) = {m}_{1 , 0} {\int }_{τ = 0}^{τ = t} \frac{{ρ}_{1} \cdot L \cdot {D}^{3}}{{12}_{η \cdot I \cdot k}^{( 11 )}} {p}_{1} ( {p}_{2} - {p}_{1} ) d τ$$

Moreover, the following applies in balloon 4:

$$\frac{{m}_{1}}{{V}_{1}} = {ρ}_{1} = \frac{{p}_{1}}{k} \qquad ( 12 )$$

Therefore, the following can be written in equation (11) for mass m₁:

$${m}_{1} = \frac{{V}_{1} \cdot {p}_{1}}{k} \qquad ( 13 )$$

The result:

$$\frac{{V}_{1}}{k} \cdot {p}_{1} ( t ) = \frac{{V}_{1}}{k} \cdot {p}_{1.} - {{\int }_{τ = 0}}^{τ = t} \frac{L \cdot {D}^{3}}{12 η \cdot l \cdot k} \cdot [ {p}_{1}^{2} - {p}_{1} {p}_{2} ] d τ \qquad ( 14 )$$

The entire equation can be shorted to V1/k. A differentiation on both sides results in:

$$\frac{{dp}_{1}}{dt} = - \frac{L \cdot {D}^{3}}{12 {V}_{1} η l} \cdot [ {p}_{1}^{2} - {p}_{1} {p}_{2} ] \qquad ( 15 )$$

This is a Bernoulli differential equation in the form:

x′=−a·x·(x−b), (16)

wherein

$$a = \frac{L \cdot {D}^{3}}{12 {V}_{1} η l} \qquad ( 17 )$$

$$b = {p}_{2} \qquad ( 18 )$$

Hereafter it should be assumed that balloon 8 is significantly larger than balloon 4:

V₂>>V₁

From this it follows that the pressure p2 in balloon 8 remains nearly, even when pressure p1 in balloon 4 briefly changes. Under this assumption, the coefficients a and b from Bernoulli differential equation (16) are constant, and the solution to the Bernoulli differential equation is:

$$x ( t ) = \frac{b}{1 - {e}^{- abt - {bc}_{1}}} \qquad ( 19 )$$

The constant of integration c₁ can be determined as follows:

$${p}_{1} ( t ) = \frac{{p}_{2}}{1 - {e}^{- {ap}_{2} t - {p}_{2} {c}_{1}}} \qquad ( 20 )$$

For t=0, the following must apply:

p₁(t)=p_{1.0} (21)

The result:

$${p}_{1.} = \frac{{p}_{2}}{1 - {e}^{- {p}_{2} {c}_{1}}} \qquad ( 22 )$$

$$\frac{{p}_{2}}{{p}_{1.}} = 1 - {e}^{- {p}_{2} {c}_{1}} \qquad ( 23 )$$

$${e}^{- {p}_{2} {c}_{1}} = \frac{{p}_{1.} - {p}_{2}}{{p}_{1.}} \qquad ( 24 )$$

$$- {p}_{2} {c}_{1} = ln \frac{{p}_{1.} - {p}_{2}}{{p}_{1.}} \qquad ( 25 )$$

$${c}_{1} = - \frac{1}{{p}_{2}} \cdot ln \frac{{p}_{1.} - {p}_{2}}{{p}_{1.}} = \frac{1}{{p}_{2}} \cdot ln \frac{{p}_{1. 0}}{{p}_{1.} - {p}_{2}} \qquad ( 26 )$$

Inserted into equation (2), this provides:

$$\frac{{p}_{1} ( t )}{{p}_{1.}} = \frac{{p}_{2}}{{p}_{1.} - [ {p}_{1.} - {p}_{2} ] \cdot {e}^{- ( π {R}^{4} {p}_{2} t ) / ( 8 {V}_{1} η l )}} \qquad ( 27 )$$

This equation is in the form:

$$\frac{{p}_{1} ( t )}{{p}_{1.}} = \frac{{p}_{2}}{{p}_{1.} - [ {p}_{1.} - {p}_{2} ] \cdot {e}^{- t / τ}} \qquad ( 28 )$$

where

T=(12·V₁·η·l)/(L·D³·p₂) (29)

The following applies for minor pressure fluctuations in balloon 4, for example:

P_{1.0}=0.9p₂

Moreover, for t=T:

e^{-t/T}=e⁻¹≈0.368=k₁

Additionally, for t=2t:

e^{-t/T}=e⁻²≈0.135=k₂

And for t=4t:

e^{-t/T}=e⁻⁴=0.018=k₄

In equation (28) this yields:

$$\frac{{p}_{1} ( t = v τ )}{0.9 {p}_{2}} = \frac{{p}_{2}}{0.9 {p}_{2} - ( 0.9 {p}_{2} - {p}_{2} ) \cdot {k}_{v}}$$

$$and$$

$$\frac{{p}_{1} ( t = v τ )}{0.9 {p}_{2}} = \frac{{p}_{2}}{( 0.9 + 0.1 \cdot {k}_{v} ) \cdot {p}_{2}}$$

The result:

$${p}_{1} ( t = τ ) = {p}_{2} \cdot \frac{0.9}{0.9 + 0.1 \cdot 0.368} \approx 0.96 \cdot {p}_{2}$$

The control deviation of approximately 0.04·p₂ remaining after t=τ corresponds to 40% of the initial deviation of 0.10··p₂.

$${p}_{1} ( t = 2 τ ) = {p}_{2} \cdot \frac{0.9}{0.9 + 0.1 \cdot 0.135} \approx 0.98 \cdot {p}_{2}$$

The control deviation of approximately 0.02·p₂ remaining after t=2τ corresponds to 20% of the initial deviation of 0.10··p₂.

$${p}_{1} ( t = 4 τ ) = {p}_{2} \cdot \frac{0.9}{0.9 + 0.1 \cdot 0.018} \approx 0.99 \cdot {p}_{2}$$

The control deviation of approximately 0.01·p₂ remaining after t=4τ corresponds to 10% of the initial deviation of 0.10··p₂.

When applied within the framework of thoracic respiration, it should be noted that a breathing cycle lasts about 3 sec. So that the cuff does not become leaky over the course of a thoracic breathing cycle, this compensation time should be tₐ=_{vT}=20 ms, wherein, with the parameter v, it is possible to choose how good the compensation should be after 20 ms.

This results in τ=20 ms/v. The minimal result to be sought for v=1 and tₐ=20 is provided as follows: From this comes:

$$τ = 20 \cdot {10}^{- 3} s = \frac{12 \cdot 5 \cdot {10}^{- 6} {m}^{3} \cdot 0.2 m \cdot 17.1 \cdot {10}^{- 6} Pas}{L \cdot {D}^{3} \cdot {10}^{5} Pa}$$

In the process, it was assumed:
V₁=5 cm³
I=20 cm
p₂=10⁵ Pa
   This results in:

L·D³=10.26·10⁻¹⁴ m⁴,

It should hereafter further be assumed that, at most, an interior opening with a maximum diameter of 10 mm, corresponding to a radius of 5 mm, is available in the tracheal tube. If one further disregards the cross-section of the outer surface of tube 3 and balloon 4, then the secondary lumen extends a maximum distance outward, and a medium radius Rm of e.g. 4.8 mm can thus be assumed. From this, it is possible to calculate a circumferential length Lm=2·n·Rm of approximately 30 mm=30·10−3 m, and from this results:

D³=10.26·10⁻¹⁴ m⁴/L

and:

D³=102.6·10⁻¹² m³/30

D³=3.42·10⁻¹² m³

D=1.5·10⁻⁴ m=0.15 mm.

The secondary lumen thus has a cross-sectional area Q2 of

Q₂=L·D=30·mm·0.15 mm=4.5 mm².

For cross-sectional area 01 of the primary lumen, D can be subtracted from the 5 mm maximum radius of the tracheal tube, and the result is 4.8 mm. This corresponds to a cross-sectional area 01 of

Q₁=4.85 mm·4.85 mm·3.14=74 mm².

The overall free cross-section Q=Qi+Q2=78.5 mm2. This means:

Q₂/Q=Q₂/(Q₁+Q₂)=4.5/78.5=0.06.

If a shorter compensation time or better compensation within this compensation time ta is required, then more stringent requirements arise for the above ratio. Accordingly, the value of 0.06 represents the absolute lower limit, which should never be undercut because this would threaten aspiration. In order to have a safety reserve, at least the following should be selected:

Q₂/Q=Q₂/(Q+Q₂)≥0.08.

Moreover, the extracorporeal supply line 7 was likewise disregarded in the above calculation, although its contribution to flow resistance is not insignificant. It is therefore recommended:

Q₂/Q=Q₂/(Q₁+Q₂)≥0.10.

If, on the other hand, one sets v=4 and ta=10 ms (i.e. the requirement that the remaining control deviation should be less than 10% after 10 ms), then the following is obtained:

$$τ = 10 ms / 4 = 25 \cdot {10}^{- 4} s = \frac{12 \cdot 5 \cdot {10}^{- 6} {m}^{3} \cdot 0.2 m \cdot 17.1 \cdot {10}^{- 6} Pas}{L \cdot {D}^{3} \cdot {10}^{5} Pa}$$

This then results in:

L·D³=82.08·10⁻¹⁴ m⁴,

D³=82.08·10⁻¹⁴ m⁴/L

or, when L=30 mm:

D³=27.4·10⁻¹² m³

D=3·10⁻⁴ m=0.3 mm.

The secondary lumen thus has a cross-sectional area Q₂ of

Q₂=L+D=30·mm·0.3 mm=9 mm².

For the cross-sectional area 01 of the primary lumen, D can be subtracted from the 5 mm maximum radius of the tracheal tube, and the result is then 4.6 mm. This corresponds to a cross-sectional area Q1 of

Q₁=4.6 mm·4.6 mm·3.14=66 mm².

The overall free cross-section Q=Q1+Q2=75 mm². This means:

Q₂/Q=Q₂/(Q₁+Q₂)=9/75=0.12.

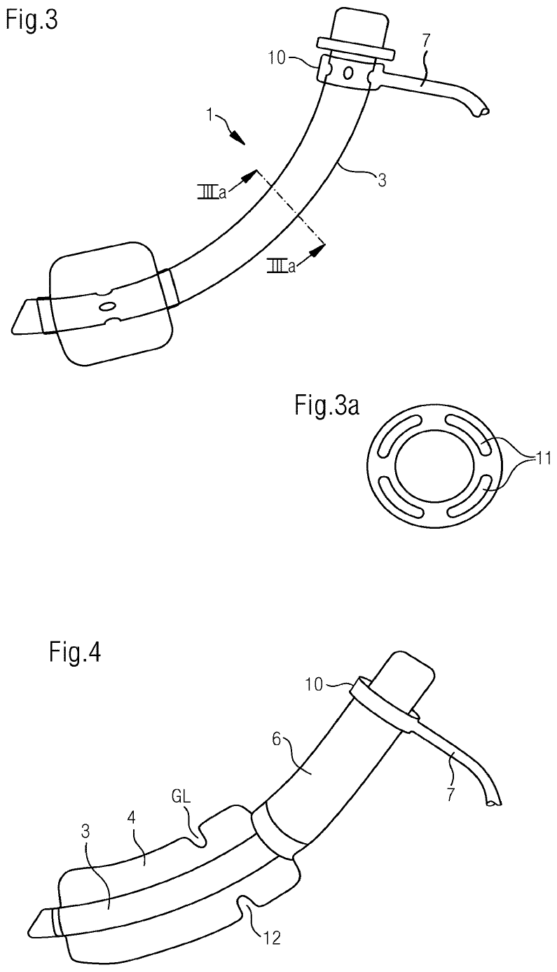

FIG. 3 describes an embodiment of the shaft body 3 that is integrated into the shaft wall, has one or more volume-supplying channels with a volume-shifting overall cross-section that corresponds in its flow mechanics with the ratios represented in FIG. 2. Here the shaft body preferably consists of multi-lumen extruded tube material which, in addition to a central lumen for ventilation, contains supply lumens disposed around said central lumen. In one such multi-lumen embodiment, the individual lumens can be bundled or combined at the proximal shaft end by an annular structure 10.

FIG. 3a shows an illustrative shaft cross-section with multi-lumen volume supply lines 11.

FIG. 4 shows an embodiment variant in which the trachea-sealing balloon segment 4 is extended proximally up to or beyond the plane of the vocal folds GL. This embodiment, in which the balloon segment that is elongated in this way protrudes proportionally from the thorax and is thus not exposed to fluctuations in thoracic pressure, permits an especially large balloon volume that is capable of developing a pressure-receiving buffer effect when a reduction in transmural force on the balloon, caused by breathing mechanics, occurs in the distal, trachea! segment of the balloon. The volume reserve created in this way also has a partial buffering effect when no external volume-compensating unit is connected to the tracheal tube.

In addition, owing to the large contact surface with the exposed tracheal, glottic and supraglottic mucous membranes, a maximally elongated migration path for secretions and pathogens contained therein is created.

To facilitate the trans-glottic positioning of the tube, the balloon can be provided with a circular constriction 12 in the region of the vocal cords GL This constriction additionally allows for the free movement of the vocal folds, largely independent of the prevailing filling pressure in the balloon.

The distal shaft segment is preferably configured as a thin-walled, single-lumen tube that is stabilized by a corrugation in the shaft wall. The distal shaft transitions in the proximal direction into a shaft segment 6 that, as described in FIG. 1, allows for a large-bore supply line to the proximal balloon segment 5. In a preferred embodiment, as described in FIG. 3a, the shaft 6 is designed with a multi-lumen profile. The multi-lumen shaft segment 6 is configured to be stable enough to serve as a bite guard, which prevents a lumen-sealing closure of the ventilation lumen.

The supplying lumens that are integrated into the shaft 6 can be bundled by a terminal element 10 at the proximal end of the tube. In turn, the connection element 7 is attached to a reservoir with a sufficiently large-bore connection.

The thin-walled, single-lumen shaft body 3 is equipped with a wavy corrugation to stabilize the shaft lumen and to permit the largely tension-free axial bending of the shaft. In the preferred embodiment, it should be possible in this way to bend the shaft from 90 to 135 degrees without relevant lumen constriction and without elastic restoring forces acting upon the tissue.

For inner shaft diameters of 7 to 10 mm in the combination of a wall thickness of ca. 0.4 mm, a Shore hardness value of 95A, a peak-to-peak wave distance of 0.5 mm and a wave amplitude of 0.75 mm, it is possible to produce a correspondingly kink-resistant lumen- and 10 flow-optimized shaft.

In the case of the corrugated embodiment of the shaft 3, when an exchangeable inner cannula is used, such as those that are conventional in tracheostomy cannulas, it is possible to use an inner cannula with a congruently corrugated profile with a corrugation that optimally conforms to the corrugation of the outer cannula and advantageously stabilizes the outer cannula.

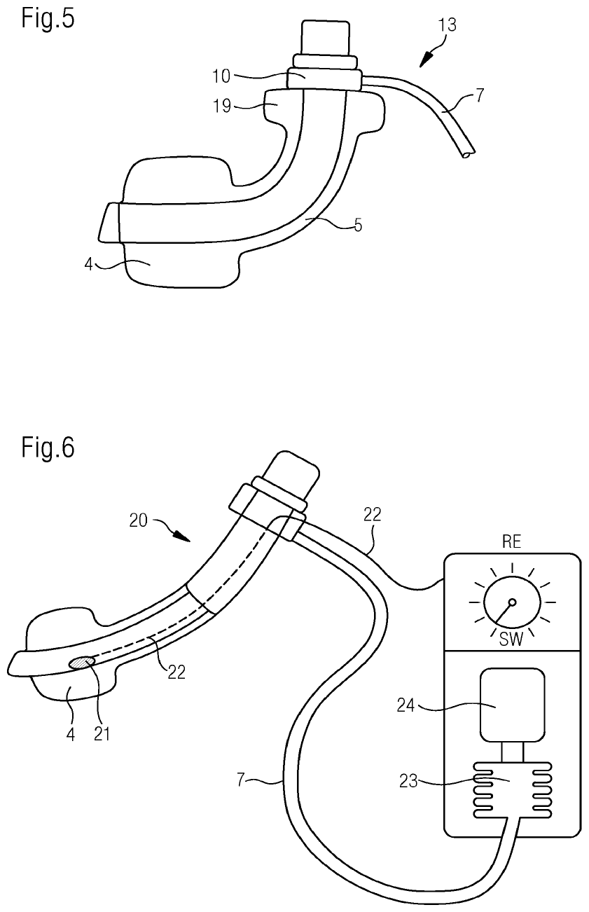

FIG. 5 shows an illustrative application of the flow-optimized embodiment of the supplying lumen to the trachea-sealing balloon element 4 in a tracheostomy cannula 13. Similar to the embodiment of trachea! tubes, the volume-supplying balloon end 5 here is led to the surgically created stoma to the trachea and applied to a connector 10 below the cannula flange. The cross-sectional area G of the supplying end 5 can be selected such that, beyond the claimed requirements of a fast volume flow, it is suitable to seal the stoma and thus prevent the escape of secretions. The proximal balloon end 19 can also advantageously be configured as a bulge-like widening, which lies sealingly against the stoma directly below the cannula flange.

FIG. 6 shows a tracheal tube 20, which is provided in the region of the trachea-sealing balloon segment 4 with a pressure-sensitive or pressure-measuring sensor element 21. In a preferred embodiment, the pressure sensor is an electronic component that relays its measurement signal to an electronically controlled regulator RE via a cable line 22. The sensor element 21 preferably consists of an absolute pressure sensor. For example, sensors based on strain gauges or piezoelectric sensors can be used.

The input signal e(t) of the regulator RE is the difference between the filling pressure actually measured by the sensor element 21 in the region of the intracorporeal sealing balloon segment 4 and a setpoint value adjustable via an extracorporeal setting control element SW.

The output signal y(t) of the regulator RE is fed as a corrective signal to a drive element 24 which acts on a bellows-like reservoir 23 in order to increase or decrease the volume thereof. The drive element 24 can consist of a step motor or can be configured as a linear magnetic drive.

The control function of the regulator RE is designed in such a way that the bellows-like reservoir 23 actuated by the drive element 24 according to the regulator output signal y(t) either shifts volume to the intracorporeal sealing balloon 4 by decreasing the volume of the bellows-like extracorporeal reservoir 23, or removes volume from the intracorporeal sealing balloon 4 by increasing the volume of the bellows-like extracorporeal reservoir 23.

The primary object of the regulator RE is to keep the actual filling pressure inside of the intracorporeal filling balloon as constant as possible at the setpoint value SW. The intracorporeal sealing balloon 4 as the controlled process unit is exposed to a disturbing signal resulting from the breathing of the patient, whereby the internal cross-section of the trachea changes. Therefore, two different conditions can be distinguished regarding the control loop:

Between two breathing cycles, the filling pressure inside of the sealing balloon 4 can be stabilized at an adjusted setpoint value SW. In this control method, the sealing balloon pressure is stabilized at a point before a mechanical breath commences and before an actual volume flow of breathing gas into the patient's lungs. This is relevant especially in patients who, for example, must expend increased breathing effort after a long period of controlled machine-assisted ventilation in order to stretch an insufficiently elastic lung in the thorax to a point that triggers a volume flow of breathing air into the lung.

Once the breathing commences, there is a phase of an “isometric” tension of the lung within the thorax and thus of the accompanying decrease in pressure within the thorax, and due to such a pressure decrease, drops in the fining pressure of the intracorporeal sealing balloon 4 can occur which can be critical to the seal. In clinically apneic patients, i.e. patients who can perform (isometric) breathing but do not produce a perceptible breathing gas stream, the described sensor technology also makes it possible to ensure intubation in a manner that prevents aspiration. In the control loop, the breathing-induced pressure fluctuations produce a disturbing signal which has to be compensated by the control loop.

Another disturbing signal may result, if sudden pressure fluctuations occur in the intracorporeal sealing balloon 4, such as when the patient changes positions or suffers a coughing attack. Then, the control loop described has to efficiently and quickly shift volume to the sealing balloon or remove volume from it in a likewise manner.

In contrast to a regulating reservoir 2, like the one described in FIG. 1 which provides a compensating reserve volume at an isobaric pressure of preferably 20 to 35 mbar, in the electronic regulation within the pressure-generating element 22 of the regulator RE it is possible to build up a pressure that briefly exceeds the tracheally uncritical sealing pressure of 20 to 35 mbar and thereby accelerate the volume flow toward the sealing balloon by means of a corresponding transient pressure gradient. The continuous measurement function of the sensor thereby ensures that the pressure in the balloon does not reach critical levels.

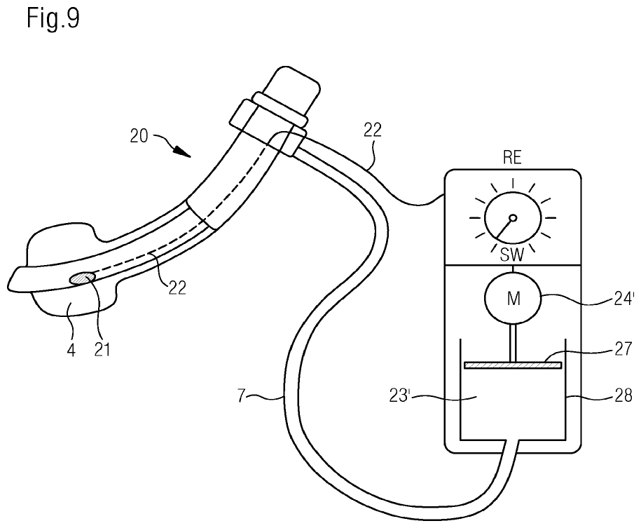

FIG. 9 shows the same or an equivalent tracheal tube 20 as FIG. 6. Especially, in the region of the trachea-sealing balloon segment 4, there is provided a pressure-sensitive or pressure-measuring sensor element 21. The measured signal of this sensor element 21 is fed via a cable 22 or via Bluetooth or by another wireless transmission to the regulator RE. Instead of the bellows-like reservoir 23, the embodiment in FIG. 9 comprises a piston-and-cylinder reservoir 23′. Thereby, a piston 27 is fed displaceable in a longitudinal direction within a cylinder 28 and completes the cylinder 28 as an enclosure of the effectively encircled volume. Thereby, the effective volume of the piston-and-cylinder reservoir 23′ is proportional to the displacement of the piston 27 within the cylinder 28, and this displacement can be controlled by a control signal y{t) transmitted to an actuator 24′ as a position control signal. In such case, the effective volume of the piston-and-cylinder reservoir 23′ is 20 proportional to the control signal y{t) generated by the control function of the regulator RE.

On the other hand, according to the Hagen-Poiseuine equation, the volume flow rate dV/dt in the pipe connection between the extracorporeal piston-and-cylinder reservoir 23′ and the intracorporeal sealing ballon 4 is proportional to the pressure difference Δp between the extracorporeal pressure Pe=KM*y(t) in the extracorporeal piston-and-cylinder reservoir 23′ and the intracorporeal pressure Pi=(1/Ks)*[e(t)+SW] measured in the intracorporeal sealing balloon 4:

dV/dt~pₑ−pᵢ=K_{M}*y(t)−(1/Ks)*[e(t)+SW],

wherein KM is the proportionality factor of the actuator element 24, and Ks is the proportionality factor of the sensor element 23′, so that the output signal s(t) of the sensor element 23′ is given as:

s(t)=K_{S}*pᵢ.

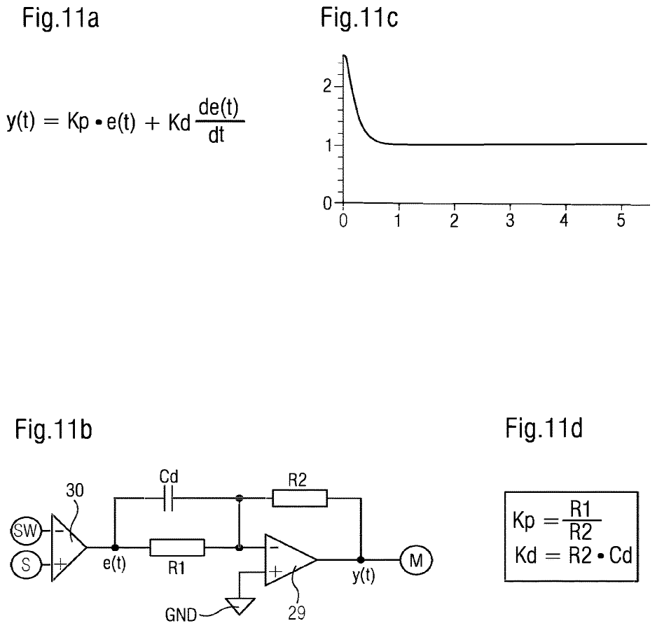

In FIG. 11 a, a preferred control function is defined, with a proportional parameter Kp and a derivative parameter Kd.

Under static conditions, the derivative term de (t)/dt is zero, and the control function simplifies to:

y(t)=Kₚ*e(t),

and this results in:

dV/dt−pₑ−pᵢ=K_{M}*Kₚ*e(t)−(1/K_{S})*[e(t)+SW]=0,

[K_{M}*Kₚ−(1/K_{S})]*e(t)=(1/K_{S})*SW.

[K_{S}*K_{M}*Kₚ−1]*e(t)=SW,

e(t)=[K_{S}*K_{M}*Kₚ−1]*SW,

pᵢ=(1/K_{S})*[e(t)+SW]=(1/K_{S})*{[K_{S}*K_{M}*Kₚ−1]⁻¹+1}*SW,

pᵢ=(1/K_{S})*{(K_{S}*K_{M}*Kₚ)/[K_{S}*K_{M}*Kₚ−1]}*SW.

pᵢ={(K_{M}*Kₚ)/[K_{S}*K_{M}*Kₚ−1]}*SW.

pᵢ=[Kₚ−1/(K_{M}*K_{S})]⁻¹*SW.

This is the stable pressure Pi inside the intracorporeal sealing balloon 4 at an adjusted setpoint value SW.

In case of a disturbation, a control function with only a proportional term Kp*e(t) will only achieve a complete compensation after a rather long period, because the desired pressure value Pi adjusted by the setpoint value SW is only reached asymptotically. To provide a rather fast compensation, an additional derivative term is provided according to the control function as disclosed in FIG. 11a.

FIG. 11 c shows the step response y(t) of such a controller. The derivative term causes a steep rise at the moment where a unit step function is applied as the input signal e(t). After a short time, the derivative portion of the controller's output signal y(t) decreases rapidly and only the constant, proportional portion of the controller's output signal y{t) remains, which is responsible for a stable 5 behavior of the closed loop.

According to a preferred embodiment, the peak of the output function y(t) at the moment where a unit step function is applied as the input signal e{t) can be more than double the constant value to which the output function y(t) of the controller approaches asymptotically, or even higher, for example more than three times of the constant value, especially more than four times of the constant value. The higher the ratio between the initial peak and the constant value is, the faster is the dynamic of the regulation, and the better is the dynamic sealing effect of the intracorporeal sealing balloon 4 even in the moment of disturbations.

On the other hand, the ratio between the between the initial peak and the constant value is strongly influenced by the ratio of Kd/Kp, which is given as:

K_{d}/Kₚ=R1*Cd.

As the resistor R1 is given by the above mentioned relation between Pi and SW, the ratio between KcJ and Kp has to adjusted via the capacitor Cd. For example, in order to reach a ratio of Ka≥2*Kp, the Capacitor Cd should be dimensioned as follows:

Cd≥2*s/R1,

or:

Cd≥5*s/R1,

or:

Cd≥10*s/R1,

with s=second.

A circuit diagram with the characteristic function according to FIG. 11 a is shown in FIG. 11b. One can see a feedback operational amplifier 29, with its positive input+is connected to ground GND, while the output signal y(t) is fed back to the negative input−via a resistor R2.

The input signal e(t) is derived via a subtracter 30 as a difference between the sensor output signal s(t) and the adjusted setpoint value SW. On the other hand, this input signal e{t) is fed via a parallel circuit of a resistor R1 and a capacitor Cd to the negative input−of the operational amplifier 29.

The parameters Kp and Kd of the characteristic function according to FIG. 11a are related to the resistors R 1, R2 and the capacitor Cd of the circuit shown in FIG. 11 b by two equations given in FIG. 11d.

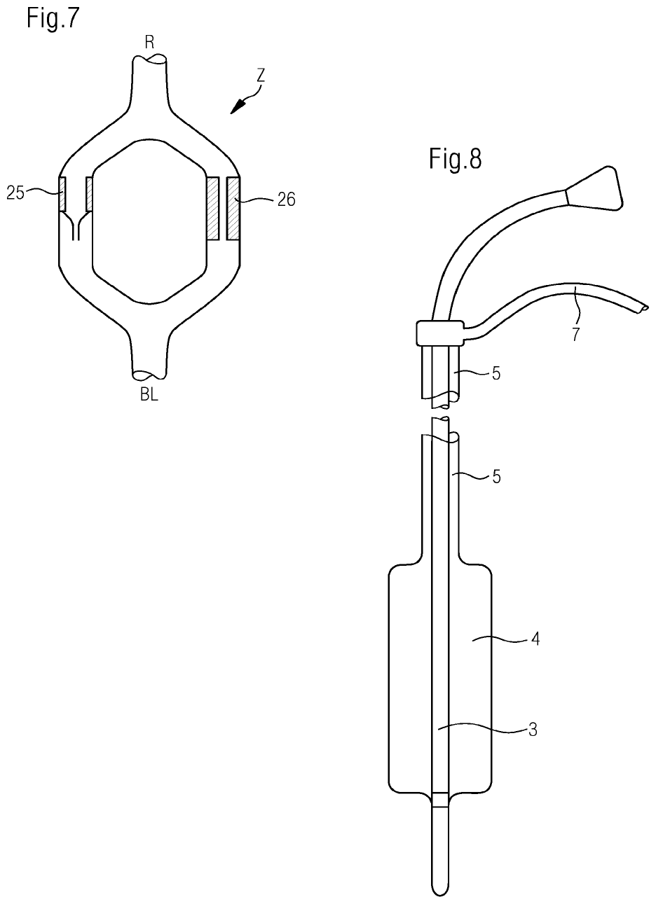

According to FIG. 7, in order to avoid larger deflations of the tracheal balloon segment or balloon 4 into the reservoir 23, 23′, which would be critical for the seal, such as those that can occur when the patient coughs or clenches, the connecting supply line Z between the balloon 4 and the regulator RE can be provided with a large-bore, flow-directing valve 25, which prevents the backflow of filling medium from the balloon BL to the reservoir R. A throttle element 26 that is not flow-directing is arranged in parallel thereto to permit the slow exchange of volume between the balloon and reservoir.

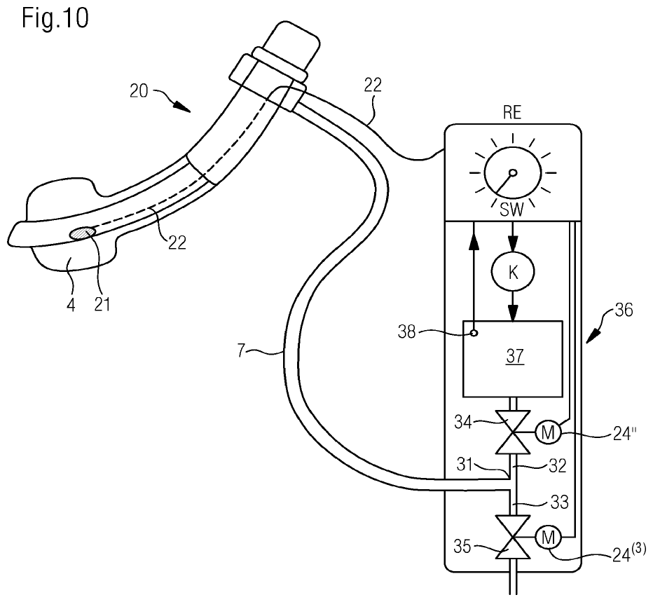

FIG. 10 discloses a still different embodiment of the invention. While the tracheal tube 20 itself and the regulator RE are the same or equivalent to those as shown in FIG. 6 or 9, especially equipped with a pressure-sensitive or pressure-measuring sensor element 21 like that disclosed in FIGS. 6 and/or 9, the bellows-like reservoir 23 of FIG. 9 and the piston-and-cylinder reservoir 23′ of FIG. 9 are replaced by another configuration:

The extracorporeal supply line 7 is connected to a branch piece 31 with at least three branches 32, 33, for example in the form of a T-piece or a Y-piece.

A valve 34, 35 is connected to each of the other branches 32, 33 of the branch piece 31, which are not connected to the extracorporeal supply line 7. Preferably, one or both valves 34, 35 are proportional valves, which can be adjusted continuously between a maximal open position and a maximal dosed position.

Each of these two valves 34, 35 is coupled to an actuator 24″, 24⁽³⁾, which in turn is controlled by the regulator RE.

One valve 35 is open to the environment, and if this valve 35 is opened, the intracorporeal sealing balloon 4 is deflated. The flow rate can be continuously controlled by the valve 35.

The other valve 34 is connected to a pressure source 36, and if this valve 34 is opened, the intracorporeal sealing balloon 4 is inflated. The flow rate can be continuously controlled by the valve 34.

The pressure source is preferably in the form of a reservoir 37 which is connected to a compressor K. In order that the pressure in the reservoir 37 can be regulated to a constant pressure value, a pressure sensor 38 is provided within the reservoir 37.

Preferably, the pressure setpoint value for the control loop for regulating the pressure Pr in the reservoir 37 is adjusted to a value 25 above the setpoint value SW for the pressure Pi inside the intracorporeal sealing balloon 4:

SW(ρᵣ)>SW(pᵢ),

for example:

SW(ρᵣ)≥1.5*SW(pᵢ)

or:

SW(ρᵣ)≥2*SW(ρᵢ),

or:

SW(pᵣ)≥3*SW(pᵢ).

or:

SW(pᵣ)≥5*SW(pᵢ).

For example, while the setpoint value SW(pi) for the pressure Pi inside the intracorporeal sealing balloon 4 may be in the range from 20 to 25 mbar, the setpoint value SW(pr) for the pressure Pr in the reservoir 37 may be at 40 mbar or above, or may be at 70 mbar or above, or may be at 100 mbar or above, or may be at 150 mbar or above.

Due to a rather dynamic control function, for example according to the PD-controller as disclosed in FIGS. 11a to 11d, the higher pressure Pr in the reservoir 37 comprises no danger for the pressure Pi in the intracorporeal sealing balloon 4.

FIG. 8 shows a similar application of the described dynamic tamponade, which allows a patient's esophagus to be sealingly closed by means of a fillable balloon element. The sealing balloon segment 4 transitions to a proximally elongated constriction 5 that defines a free gap S in the direction of the shaft element 3 for the flow-efficient shifting of a filling medium. The proximal elongation 5 of the balloon body optionally extends to or beyond the height of the mouth or nose placement The elongation 5 transitions into a tube line 7 that is configured for an efficient flow and that, in turn, is coupled to a claimed reservoir or is connected to a different claimed regulating mechanism.

With the devices described in the preceding figures for the flow-optimized shift of volume between a trachea- or esophagus-sealing balloon 4 and an extracorporeal regulating reservoir 2, seal-creating volume compensations can take place within a tracheal or esophageal balloon body within 10 to 30 milliseconds, preferably within 10 to 15 milliseconds, after the beginning of a change in intrathoracic pressure.

We claim:

1. A device for dynamically sealing intubation of a hollow organ, the device comprising:
a shaft-shaped tube configured to be inserted into the hollow organ, the shaft-shaped tube having a primary lumen that is configured to provide access through the hollow organ or to the hollow organ; and
an intracorporeal sealing balloon that surrounds a distal region of the shaft-shaped tube, wherein the intracorporeal sealing balloon is configured to operate as a cuff that dynamically seals the hollow organ,
wherein one or more secondary lumens for filling the intracorporeal sealing balloon are integrated into a wall of a proximal region of the shaft-shaped tube,
wherein at an extracorporeal filling tube that communicates with the one or more secondary lumens, a control device regulates a pressure within the intracorporeal sealing balloon such that:
when a volume of the hollow organ increases, a filling medium flows into the intracorporeal sealing balloon to increase a volume of the intracorporeal sealing balloon, and
when the volume of the hollow organ decreases, the filling medium flows out of the intracorporeal sealing balloon to decrease the volume of the intracorporeal sealing balloon,
wherein an extracorporeal reservoir is connected to the extracorporeal filling tube, and wherein the extracorporeal reservoir has a larger volume in a freely deployed state than a volume of the intracorporeal sealing balloon.

2. The device of claim 1, further comprising a valve disposed in the extracorporeal filling tube, wherein the valve is configured to permit a flow of the filling medium from the extracorporeal reservoir toward the intracorporeal sealing balloon.

3. The device of claim 2, wherein the valve is a one-way valve.

4. The device of claim 1, further comprising a pressure source connected to the extracorporeal filling tube.

5. The device of claim 4, wherein the pressure source is a source for compressed air.

6. The device of claim 4, further comprising a proportional valve arranged between the pressure source and the extracorporeal filling tube.

7. The device of claim 6, wherein the proportional valve is controlled by the control device.

8. The device of claim 4, further comprising a pressure sensor in the intracorporeal sealing balloon, and wherein the pressure sensor is configured to provide a value indicative of the pressure within the intracorporeal sealing balloon to the control device.

9. The device of claim 8, wherein the pressure sensor is combined with a temperature sensor.

10. The device of claim 8, wherein the pressure sensor is a self-calibrating sensor.

11. A device for dynamically sealing intubation of a hollow organ, the device comprising:
a shaft-shaped tube configured to be inserted into the hollow organ, the shaft-shaped tube having a primary lumen that is configured to provide access through the hollow organ or to the hollow organ; and
an intracorporeal sealing balloon that surrounds a distal region of the shaft-shaped tube, wherein the intracorporeal sealing balloon is configured to operate as a cuff that dynamically seals the hollow organ,
wherein one or more secondary lumens for filling the intracorporeal sealing balloon are integrated into a wall of a proximal region of the shaft-shaped tube,
wherein at an extracorporeal filling tube that communicates with the one or more secondary lumens, a control device regulates a pressure within the intracorporeal sealing balloon such that:
when a volume of the hollow organ increases, a filling medium flows into the intracorporeal sealing balloon to increase a volume of the intracorporeal sealing balloon, and
when the volume of the hollow organ decreases, the filling medium flows out of the intracorporeal sealing balloon to decrease the volume of the intracorporeal sealing balloon,
wherein a proximal region of the intracorporeal sealing balloon has a single secondary lumen of the one or more secondary lumens, and wherein the single secondary lumen surrounds the primary lumen.

12. The device of claim 11, further comprising a valve disposed in the extracorporeal filling tube, wherein the valve is configured to permit a flow of the filling medium from an extracorporeal reservoir toward the intracorporeal sealing balloon.

13. The device of claim 12, wherein the valve is a one-way valve.

14. The device of claim 11, further comprising a pressure source connected to the extracorporeal filling tube.

15. The device of claim 14, wherein the pressure source is a source for compressed air.

16. The device of claim 14, further comprising a proportional valve arranged between the pressure source and the extracorporeal filling tube.

17. The device of claim 16, wherein the proportional valve is controlled by the control device.

18. The device of claim 14, further comprising a pressure sensor in the intracorporeal sealing balloon, and wherein the pressure sensor is configured to provide a value indicative of the pressure within the intracorporeal sealing balloon to the control device.

19. The device of claim 18, wherein the pressure sensor is combined with a temperature sensor.

20. A method for dynamically sealing intubation of a hollow organ, the method comprising:
inserting a device into the hollow organ, the device comprising:
a shaft-shaped tube configured to be inserted into the hollow organ, the shaft-shaped tube having a primary lumen that provides access through the hollow organ or to the hollow organ; and
an intracorporeal sealing balloon that surrounds a distal region of the shaft-shaped tube, the intracorporeal sealing balloon configured to operate as a cuff that dynamically seals the hollow organ,
wherein one or more secondary lumens for filling the intracorporeal sealing balloon are integrated into a wall of a proximal region of the shaft-shaped tube,
wherein at an extracorporeal filling tube that communicates with the one or more secondary lumens, a control device regulates a pressure within the intracorporeal sealing balloon such that:
when a volume of the hollow organ increases, a filling medium flows into the intracorporeal sealing balloon to increase a volume of the intracorporeal sealing balloon, and
when the volume of the hollow organ decreases, the filling medium flows out of the intracorporeal sealing balloon to decrease the volume of the intracorporeal sealing balloon,
wherein an extracorporeal reservoir is connected to the extracorporeal filling tube, and wherein the extracorporeal reservoir has a larger volume in a freely deployed state than a volume of the intracorporeal sealing balloon.
